(19) United States
(12) Patent Application Publication
(10) Pub. No.: US 2017/0007810 A1
(43) Pub. Date: Jan. 12, 2017

(54) METHODS AND DEVICES FOR MAINTAINING AN OPEN PATHWAY IN A VESSEL

(71) Applicant: BOSTON SCIENTIFIC SCIMED, INC., Maple Grove, MN (US)

(72) Inventors: Dave Parsons, Hanover, MN (US); Robert M. Wold, Maple Lake, MN (US); Corey Tillman, Zimmerman, MN (US); Lisa Shoemaker, Champlin, MN (US); Dean A. Spencer, Golden Valley, MN (US); John B. Golden, Norton, MA (US)

(21) Appl. No.: 15/198,496

(22) Filed: Jun. 30, 2016

Related U.S. Application Data
(60) Provisional application No. 62/189,592, filed on Jul. 7, 2015.

(51) Int. Cl. A61M 29/02; A61F 2/945; A61M 25/10

(52) U.S. Cl. CPC A61M 29/02; A61M 25/1011; A61F 2/945; A61F 2/04

(57) ABSTRACT

Medical devices and methods are disclosed in the present application. In one illustrative example a medical device may comprise a catheter shaft extending from a proximal end to a distal end and the catheter shaft may include a plurality of catheter shaft lumens extending through at least a portion of the catheter shaft. The device may further include a first balloon member defining a first lumen in fluid communication with a first one of the plurality of catheter shaft lumens and a second balloon member defining a second lumen in fluid communication with a second one of the plurality of catheter shaft lumens. In some embodiments, the second balloon member may be disposed around the first balloon member and comprise a plurality of ports and a first raised portion and a second raised portion.

### CROSS-REFERENCE TO RELATED APPLICATIONS

This application claims priority under 35 U.S.C. §119 to U.S. Provisional Application Ser. No. 62/189,592, filed Jul. 7, 2015, the entirety of which is incorporated herein by reference.

### TECHNICAL FIELD

This disclosure relates to medical devices, and more particularly, to medical devices for opening constricted pathways in vessels.

### BACKGROUND

In many patients, vessels can become constricted for a variety of reasons. For example, plaque may build-up in a location within a vessel, a portion of the vessel may become calcified, or structures external to the vessel may impinge on the vessel. In some cases a medical device, such as a balloon catheter, may be used to open up the constricted portion of such vessels. A balloon member of the balloon catheter may be positioned at the constricted site and inflated to open up the vessel. Some of these procedures may additionally include positioning a stent at the treatment site to attempt to maintain an opening through the vessel at the constricted site. However, conventional stent designs may require complicated delivery procedures, encourage tissue in-growth, may block side-branch vessels, or may have other undesirable features or issues.

### SUMMARY

This disclosure relates to medical devices, and more particularly, to medical devices for opening constricted pathways in vessels and maintaining the opening. In one illustrative example, a medical device for forming a structured pathway in a vessel of a patient may comprise a catheter shaft extending from a proximal end to a distal end, the catheter shaft including a plurality of catheter shaft lumens extending through at least a portion of the catheter shaft, a first balloon member disposed proximate the distal end of the catheter shaft, the first balloon member defining a first lumen in fluid communication with a first one of the plurality of catheter shaft lumens, and a second balloon member disposed proximate the distal end of the catheter shaft and having a proximal end and a distal end, the second balloon member defining a second lumen in fluid communication with a second one of the plurality of catheter shaft lumens. In some of these examples, the second balloon member may be disposed around the first balloon member. The second balloon member may also comprise a plurality of ports disposed on an outer surface of the second balloon member. In some examples, the second balloon member may further comprise a first raised portion disposed proximate the proximal end of the second balloon member and a second raised portion disposed proximate the distal end of the second balloon member.

Additionally, or alternatively, in the above example, the first raised portion may extend outward from the outer surface of the second balloon member between about 1.0 mm and about 10.0 mm.

Additionally, or alternatively, in any of the above examples, the first raised portion may extend outward from the outer surface of the second balloon member between about 3.0 mm and about 6.0 mm.

Additionally, or alternatively, in any of the above examples, the first raised portion may extend outward from the outer surface of the second balloon member 5.0 mm.

Additionally, or alternatively, in any of the above examples, the second raised portion may extend outward from the outer surface of the second balloon member between about 1.0 mm and about 10.0 mm.

Additionally, or alternatively, in any of the above examples, the second raised portion may extend outward from the outer surface of the second balloon member between about 3.0 mm and about 6.0 mm.

Additionally, or alternatively, in any of the above examples, the second raised portion may extend outward from the outer surface of the second balloon member 5.0 mm.

Additionally, or alternatively, in any of the above examples, the first raised portion and the second raised portion may extend circumferentially around the second balloon member, each of the first raised portion and the second raised portion forming a ring-like member.

Additionally, or alternatively, in any of the above examples, the first raised portion and the second raised portion may be configured to create a seal when pressed against an interior wall of the vessel.

Additionally, or alternatively, in any of the above examples, the plurality of ports may be disposed on the outer surface of the second balloon member between the first raised portion and the second raised portion.

Additionally, or alternatively, in any of the above examples, the plurality of ports may have an open configuration and a closed configuration.

Additionally, or alternatively, in any of the above examples, the plurality of ports may transition from the closed configuration to the open configuration when a pressure difference across the plurality of ports exceeds a threshold value.

Additionally, or alternatively, in any of the above examples, the plurality of ports may allow movement of the curable liquid material across the plurality of ports from the lumen of the second balloon member to outside of the second balloon member and restrict movement of the curable liquid material across the plurality of ports from outside of the second balloon member into the lumen of the second balloon member.

Additionally, or alternatively, in any of the above examples, the plurality of ports may comprise slits.

Additionally, or alternatively, in any of the above examples, the plurality of ports may comprise one-way valves.

Additionally, or alternatively, in any of the above examples, the second one of the plurality of catheter shaft lumens may be configured to connect to a source of curable liquid material.

Additionally, or alternatively, in any of the above examples, an outer surface of the second balloon member may be resistant to adhesion by the curable liquid material.

Additionally, or alternatively, in any of the above examples, wherein the second of the plurality of lumens may be configured to transport curable liquid material from a source of curable liquid material to the lumen of the second balloon member.

Additionally, or alternatively, in any of the above examples, the medical device may further comprise a pressure source for delivering the curable liquid material through the second one of the plurality of catheter shaft lumens and out the plurality of ports.

Additionally, or alternatively, in any of the above examples, the curable liquid material may comprise cyanoacrylate.

Additionally, or alternatively, in any of the above examples, wherein the curable liquid material may comprise a two-part epoxy.

Additionally, or alternatively, in any of the above examples, an interior surface of the second one of the plurality of catheter shaft lumens may be resistant to adhesion by the curable liquid material.

Additionally, or alternatively, in any of the above examples, the medical device may further comprise a mixing feature disposed within the second one of the plurality of catheter shaft lumens.

Additionally, or alternatively, in any of the above examples, the medical device may further comprise a light source disposed proximate the first balloon member and the second balloon member.

Additionally, or alternatively, in any of the above examples, the medical device may further comprise a heat source disposed proximate the first balloon member and the second balloon member.

Additionally, or alternatively, in any of the above examples, the medical device may further comprise an RF source disposed proximate the first balloon member and the second balloon member.

Additionally, or alternatively, in any of the above examples, the medical device may be configured for use in the biliary tract.

In another illustrative example, a method of forming a structured pathway in a vessel of a patient may comprise positioning a medical device at a treatment site within the vessel, the medical device comprising: a catheter shaft extending from a proximal end to a distal end, the catheter shaft including a plurality of catheter shaft lumens extending through at least a portion of the catheter shaft, a first balloon member disposed proximate the distal end of the catheter shaft, the first balloon member defining a first lumen in fluid communication with a first one of the plurality of catheter shaft lumens, and a second balloon member disposed proximate the distal end of the catheter shaft and having a proximal end and a distal end, the second balloon member defining a second lumen in fluid communication with a second one of the plurality of catheter shaft lumens. In some examples, the second balloon member may be disposed around the first balloon member, the second balloon member may comprise a plurality of ports disposed on an outer surface of the second balloon member, and the second balloon member may comprise a first raised portion disposed proximate the proximal end of the second balloon member and a second raised portion disposed proximate the distal end of the second balloon member. In some additional examples, the method may further comprise inflating the first balloon member to dilate a portion of the vessel, delivering a curable liquid material to the lumen of the second balloon member, the curable liquid material exiting the lumen of the second balloon member through the plurality of ports, and maintaining the positioning of the medical device while the curable liquid material solidifies, the solidified curable material forming a structure to maintain an opening through the vessel. In still some additional examples, the method may further comprise deflating the first balloon member to separate the first balloon member from the solidified curable material.

Additionally, or alternatively, in any of the above examples, the method may further comprise delivering one or more of light, heat, and RF energy to the curable liquid material to solidify the curable liquid material.

Additionally, or alternatively, in any of the above examples, when the first balloon member is inflated, the first raised portion and the second raised portion may form a seal with an inner wall of the vessel and prevent the curable liquid material exiting the plurality of ports from migrating proximal of the first raised portion and distal of the second raised portion.

Additionally, or alternatively, in any of the above examples, the vessel may be in the biliary tract.

In still another illustrative example, a medical device for forming a structured pathway in a vessel of a patient may comprise a catheter shaft extending from a proximal end to a distal end, the catheter shaft including a plurality of catheter shaft lumens extending through at least a portion of the catheter shaft, a balloon member disposed proximate the distal end of the catheter shaft, the first balloon member defining a first lumen in fluid communication with a first one of the plurality of catheter shaft lumens, and a port disposed proximate the balloon member, the port being in fluid communication with a second one of the plurality of catheter shaft lumens, the port further configured for excreting a curable liquid material.

Additionally, or alternatively, in any of the above examples, the port may comprise a nozzle.

Additionally, or alternatively, in any of the above examples, the second one of the plurality of catheter shaft lumens may extend distal of the balloon member.

Additionally, or alternatively, in any of the above examples, the nozzle has a closed configuration and an open configuration, and wherein the nozzle may be configured to spray a curable liquid material when in the open position.

Additionally, or alternatively, in any of the above examples, the nozzle may transition from the closed configuration to the open configuration when the liquid curable material is delivered to the nozzle at a pressure greater than a threshold pressure.

Additionally, or alternatively, in any of the above examples, the nozzle may be configured such that, when in the open configuration, the liquid curable material exits the nozzle in a fan-shaped spray, the fan shape defining an angle between about 30 degrees and 360 degrees.

Additionally, or alternatively, in any of the above examples, the fan shape may define a spray angle of between about 90 degrees and 180 degrees.

Additionally, or alternatively, in any of the above examples, the fan shape may define a spray angle of about 360 degrees.

Additionally, or alternatively, in any of the above examples, the liquid curable material may exit the nozzle to form an angle of between about 0 degrees to 60 degrees with the nozzle.

Additionally, or alternatively, in any of the above examples, the liquid curable material may exit the nozzle to form an angle of 90 degrees with the nozzle.

Additionally, or alternatively, in any of the above examples, the nozzle may comprise a heating element.

Additionally, or alternatively, in any of the above examples, the nozzle may have a plurality of ports.

Additionally, or alternatively, in any of the above examples, the second one of the plurality of catheter shaft lumens may extend through the balloon member.

Additionally, or alternatively, in any of the above examples, the balloon member may be a first balloon member, and the medical device may further comprise a second balloon member having a proximal end and a distal end, the second balloon member comprising a first raised portion disposed proximate the proximal end of the second balloon member and a second raised portion disposed proximate the distal end of the second balloon member.

Additionally, or alternatively, in any of the above examples, the second balloon member may be disposed around the first balloon member.

Additionally, or alternatively, in any of the above examples, the second one of the plurality of catheter shaft lumens may be in fluid communication with the interior of the second balloon member.

Additionally, or alternatively, in any of the above examples, the port may comprise a plurality of ports, and the plurality of ports may be disposed on the surface of the second balloon member.

Additionally, or alternatively, in any of the above examples, the medical device may further comprise a plurality of ports disposed between the first raised portion and the second raised portion.

Additionally, or alternatively, in the above example, the first raised portion may extend outward from the outer surface of the second balloon member between about 1.0 mm and about 10.0 mm.

Additionally, or alternatively, in any of the above examples, the first raised portion may extend outward from the outer surface of the second balloon member between about 3.0 mm and about 6.0 mm.

Additionally, or alternatively, in any of the above examples, the first raised portion may extend outward from the outer surface of the second balloon member 5.0 mm.

Additionally, or alternatively, in any of the above examples, the second raised portion may extend outward from the outer surface of the second balloon member between about 1.0 mm and about 10.0 mm.

Additionally, or alternatively, in any of the above examples, the second raised portion may extend outward from the outer surface of the second balloon member between about 3.0 mm and about 6.0 mm.

Additionally, or alternatively, in any of the above examples, the second raised portion may extend outward from the outer surface of the second balloon member 5.0 mm.

Additionally, or alternatively, in any of the above examples, the first raised portion and the second raised portion may extend circumferentially around the second balloon member, each of the first raised portion and the second raised portion forming a ring-like member.

Additionally, or alternatively, in any of the above examples, the first raised portion and the second raised portion may be configured to create a seal when pressed against an interior wall of the vessel.

Additionally, or alternatively, in any of the above examples, the vessel may be in the biliary tract.

In another example, a medical device for forming a structured pathway in a vessel of a patient may comprise a catheter shaft extending from a proximal end to a distal end, the catheter shaft including a plurality of catheter shaft lumens extending through at least a portion of the catheter shaft, a balloon member disposed proximate the distal end of the catheter shaft, the first balloon member defining a first lumen in fluid communication with a first one of the plurality of catheter shaft lumens, and a port disposed distal of the balloon member, the port being in fluid communication with a second one of the plurality of catheter shaft lumens. In some additional examples, the medical device may further comprise a nozzle disposed at the port.

Additionally, or alternatively, in any of the above examples, the nozzle may have a closed configuration and an open configuration, and the nozzle may be configured to spray a liquid curable material when in the open position.

Additionally, or alternatively, in any of the above examples, the nozzle may transition from the closed configuration to the open configuration when the liquid curable material exerts a pressure on the nozzle greater than a threshold pressure.

Additionally, or alternatively, in any of the above examples, the nozzle may be configured such that, when in the open configuration, the liquid curable material exits the nozzle in a fan-shaped spray, the fan shape defining an angle between about 30 degrees and 360 degrees.

Additionally, or alternatively, in any of the above examples, the fan shape may define a spray angle of between about 90 degrees and 180 degrees.

Additionally, or alternatively, in any of the above examples, the fan shape may define a spray angle of about 360 degrees.

Additionally, or alternatively, in any of the above examples, the liquid curable material may exit the nozzle to form an angle of between about 0 degrees to 60 degrees with the nozzle.

Additionally, or alternatively, in any of the above examples, the liquid curable material may exit the nozzle to form an angle of 90 degrees with the nozzle.

Additionally, or alternatively, in any of the above examples, the nozzle may further comprise a heating element.

Additionally, or alternatively, in any of the above examples, the nozzle may comprise a plurality of ports.

Additionally, or alternatively, in any of the above examples, the second one of the plurality of catheter shaft lumens may extend through the balloon member.

Additionally, or alternatively, in any of the above examples, the medical device may further comprise a heating element.

Additionally, or alternatively, in any of the above examples, the medical device may further comprise a light source.

Additionally, or alternatively, in any of the above examples, the medical device may further comprise an RF energy source.

Additionally, or alternatively, in any of the above examples, the second one of the plurality of catheter shaft lumens may be connected to a source of curable liquid material.

Additionally, or alternatively, in any of the above examples, the medical device may further comprise a mixing element disposed within the second one of the plurality of catheter shaft lumens.

Additionally, or alternatively, in any of the above examples, the medical device may be configured for use in the biliary tract.

In another illustrative example, a method for forming a structured pathway in a vessel may comprise positioning a medical device at a treatment site within the vessel, the medical device comprising: a catheter shaft extending from a proximal end to a distal end, the catheter shaft including a plurality of catheter shaft lumens extending through at least a portion of the catheter shaft, a balloon member disposed proximate the distal end of the catheter shaft, the first balloon member defining a first lumen in fluid communication with a first one of the plurality of catheter shaft lumens, and a port disposed distal of the balloon member, the port being in fluid communication with a second one of the plurality of catheter shaft lumens, the port further configured for excreting a liquid curable material. In some examples, the method may further comprise inflating the balloon member to dilate a portion of the vessel. In still some additional examples, the method may further comprise retracting the balloon member through the dilated portion of the vessel while expelling a curable liquid material from the port and onto an inner surface of the vessel.

Additionally, or alternatively, in any of the above examples, the method may further comprise deflating the balloon member.

Additionally, or alternatively, in any of the above examples, the method may further comprise rotating the balloon member while retracting the balloon member.

Additionally, or alternatively, in any of the above examples, the method may further comprise extending the balloon member back through the dilated portion of the vessel while expelling the curable liquid material from the port and onto the inner surface of the vessel.

Additionally, or alternatively, in any of the above examples, the method may further comprise introducing one or more of light, heath, and RF energy to the curable liquid material to harden the material.

Additionally, or alternatively, in any of the above examples, the vessel may be in the biliary tract.

The above summary of the present disclosure is not intended to describe each embodiment or every implementation of the present disclosure. Advantages and attainments, together with a more complete understanding of the disclosure, will become apparent and appreciated by referring to the following detailed description and claims taken in conjunction with the accompanying drawings.

### BRIEF DESCRIPTION OF THE DRAWINGS

The disclosure may be more completely understood in consideration of the following detailed description of various embodiments in connection with the accompanying drawings, in which:

FIG. 1 is a side plan view of a catheter in accordance with various embodiments of the present disclosure;

FIG. 2 is an internal view of a balloon member of the catheter of FIG. 1;

FIG. 3 is a side view of an elongate shaft of the catheter of FIG. 1 including multiple lumens;

FIG. 4 is an internal view of a balloon member of the catheter of FIG. 1 including an energy delivery element;

FIG. 5A is side view of a balloon member of a catheter in accordance with various embodiments of the present disclosure positioned in a biliary tract vessel;

FIG. 5B is an internal view of the balloon member of FIG. 5A in a biliary tract vessel;

FIG. 5C is an internal view of the balloon member of FIG. 5A in an inflated state;

FIG. 5D is another internal view of the balloon member of FIG. 5A in a biliary tract vessel;

FIG. 5E is internal view of the balloon member of FIG. 5A in a biliary tract vessel including deposited curable material;

FIG. 5F is internal view of the balloon member of FIG. 5A in a biliary tract vessel including deposited curable material;

FIG. 6 is a side plan view of a catheter including a balloon member and a nozzle in accordance with various embodiments of the present disclosure;

FIG. 7A is a depiction of the balloon member of FIG. 6 with the nozzle in a closed position;

FIG. 7B is a depiction of the balloon member of FIG. 6 with the nozzle in an open position;

FIG. 8 is an end view of the balloon member and nozzle of FIG. 6;

FIG. 9 is a side view of the balloon member and nozzle of FIG. 6;

FIG. 10A is side view of a balloon member of a catheter in accordance with various embodiments of the present disclosure positioned in a biliary tract vessel;

FIG. 10B is a side view of the balloon member of FIG. 10A in an inflated state in the biliary tract vessel;

FIG. 10C is another side view of the balloon member of FIG. 10A in the biliary tract vessel with the biliary tract vessel dilated;

FIG. 10D is another side view of the balloon member of FIG. 10A in the biliary tract vessel including deposited curable material; and

FIG. 10E is another side view of the balloon member of FIG. 10A in the biliary tract vessel including deposited curable material.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended to be only exemplary. Selected features of any illustrative embodiments may be incorporated into any other described embodiments unless clearly stated to the contrary.

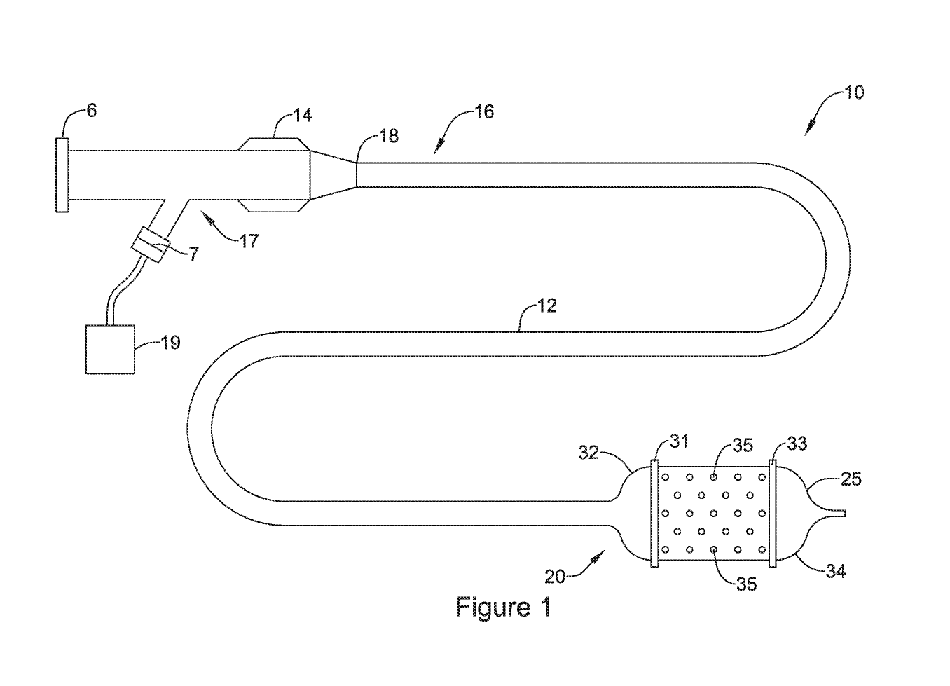

FIG. 1 shows an exemplary catheter 10 in accordance with various embodiments of the present disclosure. In some cases, catheter 10 may be a guide or diagnostic catheter, and may have a length and an outside diameter appropriate for its desired use, for example, to enable biliary tract insertion and navigation. For example, when catheter 10 is adapted as a guide catheter, catheter 10 may have a length of about 20-250 cm and an outside diameter of approximately 1-10 French, depending upon the desired application. In some cases, catheter 10 may be a microcatheter that is adapted and/or configured for use within small anatomies of the patient. For example, catheter 10 may be used to navigate to targets sites located in tortuous and narrow vessels such as, for example, to sites within the neurovascular system, certain sites within the coronary vascular system, to sites within the peripheral vascular system such as superficial femoral, popliteal, or renal arteries, or any number of locations within the biliary tract. In some cases, the target site is a neurovascular site and may be located within a patient's brain, which is accessible only via a tortuous vascular path. However, it is contemplated that the catheter may be used in other target sites within the anatomy of a patient. An exemplary catheter that may be utilized in accordance with the various embodiments as described herein is shown and described in U.S. Pat. No. 8,182,465, which is incorporated herein by reference in its entirety for all purposes.

As shown in FIG. 1, catheter 10 can include elongate catheter shaft 12. Elongate shaft 12 may generally extend from proximal portion 16 and proximal end 18 toward distal portion 20. Although elongate shaft 12 may have a circular cross-sectional shape, it should be understood that elongate shaft 12 can have other cross-sectional shapes or combinations of shapes without departing from the scope of the disclosure. For example, the cross-sectional shape of the generally tubular elongate shaft 12 may be oval, rectangular, square, triangular, polygonal, and the like, or any other suitable shape, depending upon the desired characteristics. In some cases, manifold 14 may be connected to proximal end 18 of elongate shaft 12.

The manifold may include hub 17 and/or other structures to facilitate connection to other medical devices (e.g., syringe, stopcocks, Y-adapter, etc.) and to provide access to one or more lumens defined within elongate shaft 12. In some cases, hub 17 may include ports 6 and 7 which provide individual access to one or more lumens extending through at least a portion of catheter 10. Some example lumens that may extend through catheter 10 may include at least one guidewire lumen, one or more inflation lumens, and, in some cases, a lumen for delivering a curable material, whether in a solid form or a liquid form. The lumens that do extend through catheter 10 may terminate at or near distal portion 20 of elongate shaft 12, as will be described with respect to other figures. However, in other cases, hub 17 may have a single port, three ports, or any other number of ports. Manifold 14 may also include a strain relief portion adjacent proximal end 18 of elongate shaft 12.

Distal portion 20 of elongate shaft 12 may include balloon member 25, as shown in FIG. 1. Balloon member 25 may be an inflatable balloon and may have a lumen that is connected to one or more of the lumens extending through elongate shaft 12. It should be understood that the depiction of balloon member 25 in FIG. 1 is only an example. In other embodiments, balloon member 25 may have any shape, and in some embodiments may comprise two or more balloons. Balloon member 25 may additionally have first raised portion 31 disposed proximate proximal end 32 of balloon member 25. Balloon member 25 may further include second raised portion 33 disposed proximate distal end 34 of balloon member 25. In some embodiments, first raised portion 31 and second raised portion 33 extend all the way around balloon member 25, for example forming ring-like structures.

In some additional embodiments, balloon member 25 may further include ports 35 disposed on the outer surface of balloon member 25. Ports 35 may fluidly connect a lumen of balloon member 25 with the exterior of balloon member 25. As mentioned, the lumen of balloon member 25 may be connected to one or more of the lumens extending through elongate shaft 12. Accordingly, material may be introduced at one or more of ports 6 and 7 and be delivered to ports 35 through one or more lumens of elongate shaft 12 and through balloon member 25. Ports 35 may regulate the flow of material out of, and in some embodiments into, balloon member 25. As one example, ports 35 may act as one-way valves, only allowing material to flow in one direction across the surface of balloon member. In at least some embodiments, ports 35 may only be disposed between first raised portion 31 and second raised portion 33, as shown in FIG. 1.

Catheter 10 may additionally be connected to reservoir 19. Reservoir 19 may be connected to a port of catheter 10, such as port 7, which connects with one or more lumens of catheter 10. Accordingly, reservoir 19 may contain material to be delivered to balloon member 25 through one or more lumens of catheter 10. In some cases, the material may be delivered to ports 35, which regulate the transfer of the material to outside of balloon member 25. Reservoir 19 may additionally contain, or be connected to, a pressure source for actively delivering the material stored in reservoir 19 to catheter 10 and balloon member 25. For instance, reservoir 19 may contain, or be connected to, an electric pump that pumps the material into catheter 10. In other embodiments, reservoir 19 may contain, or be connected to, a manual pump that a user may employ to pump the material into catheter 10. In some embodiments, reservoir 19 may represent a syringe filled with the material, where application of force to the syringe plunger pushes the material into and through catheter 10. These are just some examples. Other embodiments may have different sources of pressure.

In accordance with techniques described herein in more detail, catheter 10, including balloon member 25, may be used to form a structure in-situ. For instance, reservoir 19 may contain a curable material that may be delivered to balloon member 25 through a lumen of catheter 10 that is configured to transport the curable material. In some of these embodiments, the curable material may be in liquid form. For instance, the liquid curable material may be any thermo-setting or UV curable polymer that is safe for use within a human body. Other examples of curable liquid materials include two-part epoxies which, when mixed together, form a hardened structure. Some specific examples include cyanoacrylate or other biocompatible, medical adhesives. In some additional examples, the curable material may initially be in a solid form. The solid curable material may be melted during delivery to balloon member 25, for instance before being delivered into a lumen of catheter 10.

Catheter 10 may be maneuvered so as to position balloon member 25 within a passageway of a vessel of a patient. Although described herein with respect to a biliary vessel, it should be understood that the devices of this disclosure may be used in any constricted vessel to dilate the vessel and from a structure in-situ to prevent the vessel from re-constricting. Accordingly, once balloon member 25 is disposed at a restriction site, balloon member 25 may be inflated to dilate the constriction and curable material may be delivered to balloon member 25. The curable material may exit through ports 35 adjacent to the biliary vessel wall. The curable material may then harden into a solid tubular structure that maintains patency of the biliary vessel.

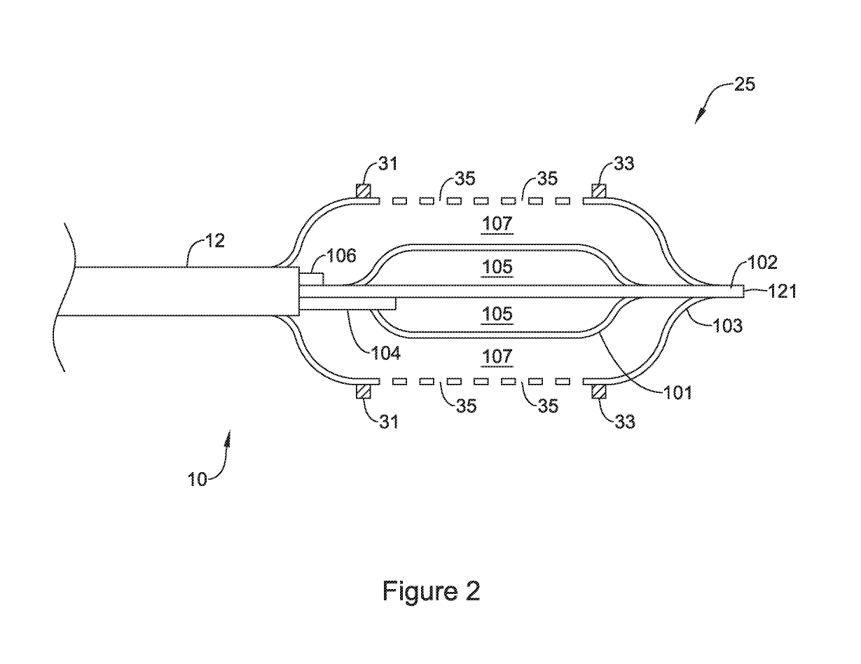

FIG. 2 depicts an internal view of balloon member 25, with first balloon member 101 and second balloon member 103 in cross-section. In the embodiment of FIG. 2, balloon member 25 is shown comprising first balloon member 101 having first balloon lumen 105 and second balloon member 103 having second balloon lumen 107. In these embodiments, second balloon member 103 may be disposed around first balloon member 101. For instance, first balloon member 101 may be entirely contained within the second balloon lumen 107.

FIG. 2 additionally depicts elongate shaft 12 with three distinct delivery lumens, guidewire lumen 102 and delivery lumens 104 and 106. Guidewire lumen 102 may extend through elongate shaft 12, and catheter 10 more generally. As shown in FIG. 2, guidewire lumen 102 may additionally extend through balloon member 25, including both first balloon member 101 and second balloon member 103. Guidewire lumen 102 may connect to one of ports 6 or 7, or another port of catheter 10. Guidewire lumen 102 may also have distal port 131 that is disposed distal of balloon member 25, as shown in FIG. 2. Accordingly, catheter 10 may be delivered to a treatment site over-the-wire. In these embodiments, a guidewire may first be threaded through a patient. Once the distal end of the guidewire is located at the treatment site, the proximal end of the guidewire may be inserted into distal port 131, and catheter 10 may then be advanced over the guidewire until balloon 25 is located proximate the distal end of the guidewire at the treatment site. Although depicted as being disposed distal of balloon member 25, in other embodiments, distal port 131 may be disposed somewhere on balloon member 25, or, in still other embodiments, proximal of balloon member 25 on elongate shaft 12.

In embodiments where catheter 10 includes delivery lumen 104, delivery lumen 104 may extend through elongate shaft 12, and catheter 10 more generally. Delivery lumen 104 may fluidly connect first balloon lumen 105 to one of ports 6, 7, or another port of catheter 10. In some embodiments, delivery lumen 104 may be an inflation lumen. For instance, delivery lumen 104 may be configured to deliver inflation media to first balloon lumen 105 to inflate first balloon member 101 or siphon inflation media from first balloon lumen 105 to deflate first balloon member 101. In these embodiments delivery lumen 104 may additionally be connected to a source of inflation media and a pressure source. The pressure source may be capable of operating in a positive or negative manner such that the pressure source may deliver inflation media into delivery lumen 104 to inflate first balloon member 101 and to siphon inflation media from first balloon member 101 through delivery lumen 104. As a few examples, the pressure source may be an electric or manual pump, or any other commonly used device for delivering inflation media through a catheter.

In embodiments where catheter 10 includes delivery lumen 106, delivery lumen 106 may extend through elongate shaft 12, and catheter 10 more generally. Delivery lumen 106 may fluidly connect second balloon lumen 107 to one of ports 6, 7, or another port of catheter 10. In some embodiments, delivery lumen 106 may configured to deliver a curable material, whether in liquid or solid form, to second balloon lumen 107. For instance, an interior surface of delivery lumen 106 may be configured to be adhesion resistant to the curable material. Alternatively, or additionally, delivery lumen 106 may have a wall thickness designed to withstand the pressures required to deliver the curable material through delivery lumen 106 and into second balloon lumen 107. In these embodiments delivery lumen 106 may additionally be connected to a source of curable material and a pressure source. The pressure source may be able to deliver the curable material into and through delivery lumen 106 to second balloon lumen 107. As a few examples, where the curable material is a liquid curable material, the pressure source may be an electric or manual pump, or any other commonly used device for delivering inflation media through a catheter. In at least some embodiments, the curable material may be delivered through delivery lumen 106 in a solid form. In these embodiments, the pressure source may comprise a mechanism that exerts a force to extend the solid curable material into delivery lumen 106.

First balloon member 101 may be configured as a dilation balloon. For example, first balloon member 101 may be configured to withstand predetermined amounts of internal pressures, and, in some embodiments, have a known outer diameter at known internal pressures. In this way, a user of catheter 10 with balloon member 25 may dilate a constricted vessel to a known diameter. For example once balloon member is disposed at a constricted treatment side, a user may deliver inflation media to first balloon member 101 through delivery lumen 104. The inflation media may cause first balloon member 101 to inflate and expand against the constricted vessel. As additional inflation media is delivered to first balloon member 101, first balloon member 101 may continue to expand and widen the constricted vessel to form a patent pathway through the vessel.

As depicted in FIGS. 1 and 2, second balloon member 103 may include first raised portion 31 and second raised portion 33. First raised portions 31 may extend generally radially outward from the outer surface of second balloon member 103. In some embodiments, first raised portion 31 may extend outward from second balloon member 103 between about 1.0 mm and about 10.0 mm. In more specific embodiments, first raised portion 31 may extend outward from second balloon member 103 between about 3.0 mm and about 6.0 mm. In at least some embodiments, first raised portion 31 may extend outward from second balloon member 103 about 5.0 mm. In a similar manner to first raised portion 31, in some embodiments second raised portion 33 may extend outward from second balloon member 103 between about 1.0 mm and about 10.0 mm, or between about 3.0 mm and about 6.0 mm in more specific embodiments. In at least some embodiments, second raised portion 33 may extend outward from second balloon member 103 about 5.0 mm. Although first raised portion 31 and second raised portion 33 may extend outward from second balloon member 103 the same distance, in at least some embodiments, first raised portion 31 and second raised portion 33 may extend outward from second balloon member 103 different distances. Additionally, in at least some embodiments, first raised portion 31 and/or second raised portion 33 may extend outward from second balloon member 103 different distances at different points on first raised portion 31 and/or second raised portion 33. Each of first raised portion 31 and second raised portion 33 may generally extend outward from second balloon member 103 around the entire circumference of second balloon member 103, so that first raised portion 31 and second raised portion 33 form ring-like structures disposed around second balloon member 103. Although, in some additional embodiments, first raised portion 31 and/or second raised portion 33 may not extend outward away from second balloon member 103 around the entire circumference of second balloon member 103. As will be discussed in more detail below, first raised portion 31 and second raised portion 33 may be configured to form a seal with a vessel when pressed against a vessel, thereby creating a compartment between first raised portion 31, second raised portion 33, and the wall of the vessel.

Additionally, or alternatively, to the embodiments described above, second balloon member 103 may include ports 35. As shown in FIG. 2, ports 35 may fluidly connect second balloon lumen 107 to outside of second balloon member 103. For example, ports 35 may regulate passage of material from inside of second balloon lumen 107 to outside of second balloon member 103 and from outside of second balloon member 103 to inside second balloon lumen 107.

In some embodiments including ports 35, ports 35 may act as one-way valves. For instance, ports 35 may allow passage of material from inside of second balloon lumen 107 to outside of second balloon member 103, but may prevent passage of material from outside of second balloon member 103 to inside of second balloon lumen 107. Accordingly, in these embodiments, curable material delivered to second balloon lumen 107 may pass through ports 35 and be delivered external to second balloon member 103. Ports 35 may be made through any suitable means. For example, ports 35 may be post-formed in second balloon member 103 by puncturing second balloon member 103 with one or more thin, needle-like puncture members. In at least some of these embodiments, the puncture members may puncture second balloon member 103 from the inside. For instance, before second balloon member 103 is attached to catheter 10, the puncture members may puncture through second balloon member 103 from what will be the inside of second balloon member 103 so that material of second balloon member 103 is pushed outward at the puncture sites to form ports 35. This may work to create ports 35 as one-way valves such that, once second balloon member 103 is attached to catheter 10, ports 35 allow passage of material from inside second balloon lumen 107 to outside of second balloon member 103 but restrict passage of material from outside of second balloon member 103 into second balloon lumen 107. In other examples, ports 35 may be longitudinal and/or lateral slits created by one or more cutting members. In at least some examples, ports 35 may be holes that allow for free flow of material from inside of second balloon lumen 107 to outside of second balloon member 103 and from outside of second balloon member 103 to inside second balloon lumen 107. In still other embodiments, second balloon member 103 may not include distinct ports 35. Rather, second balloon member 103 may be semi-permeable, and selectively allow passage material out of, and in some embodiments into, second balloon lumen 107.

In some embodiments, it may be desirable to further control the flow of material through ports 35, for instance from inside second balloon lumen 107 to outside of second balloon member 103. In these embodiments, ports 35 may have a closed configuration and an open configuration. In these embodiments, ports 35 may be configured to transition from their closed state to their open state when pressure inside second balloon lumen 107 reaches a threshold pressure. Example values for the threshold pressure may range from about 0.25 psi (1.72 kPa) to about 1.25 psi (8.62 kPa). Configuring ports 35 to open in this manner may allow for more controlled release of the curable material through ports 35 than in other embodiments.

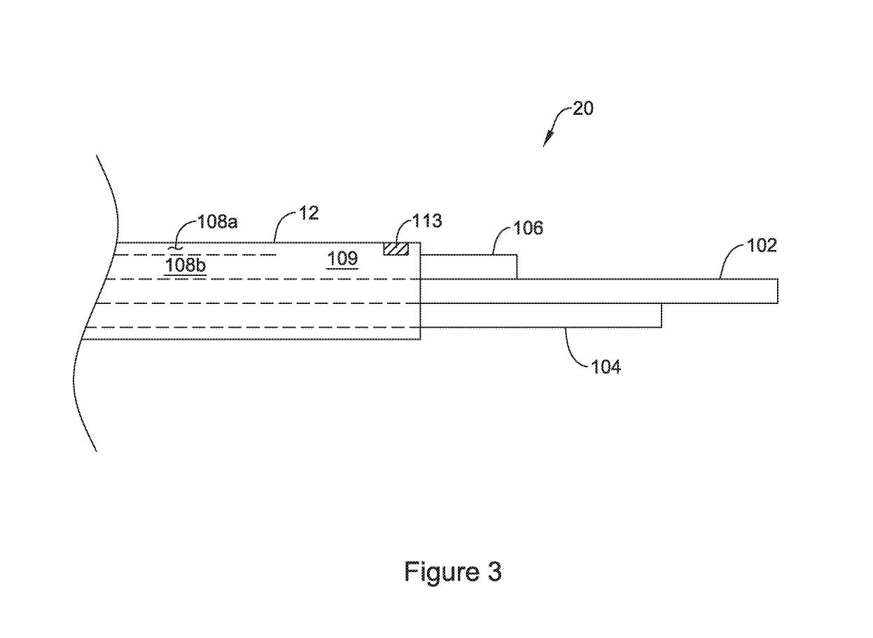

FIG. 3 depicts a close-up of elongate shaft 12, including lumens 102, 104, and 106 extending partially through elongate shaft 12. In at least some embodiments of the present disclosure, the curable material may comprise two separate components that, when mixed together, undergo a chemical reaction and solidify, sometime referred to as curing. Accordingly, in these embodiments, delivery shaft 106 may comprise sub-lumens 108a and 108b, along with a mixing region 109 to control when the two components mix together begin to cure. In these embodiments, a first one of the curable material components may be delivered proximate distal end 18 of catheter 10 in first sub-lumen 108a while the second one of the curable material components may be delivered proximate distal end 18 of catheter 10 in second sub-lumen 108b. Before connecting with second balloon lumen 107, sub-lumens 108a and 108b may merge forming mixing region 109. In mixing region 109, the two components of the curable material may mix together and begin to cure. The specific components, or the ratio of the components, may be chosen to have a curing time suitable to allow for the mixture to be delivered through ports 35 before fully curing and hardening. Additionally, in embodiments where the curing reaction is exothermic, the specific components, or the ratio of the components, may be chosen so that the reaction does not cause damage to the patient. Further, although FIG. 3 depicts delivery lumen 106 as having two sub-lumens 108a, 108b, in other embodiments, delivery lumen 106 may have more than two sub-lumens for curable materials that have more than three mixing components, or where additional material aside from the curable material is delivered to second balloon lumen 107.

Additionally, or alternatively, in some embodiments, mixing region 109 may comprise one or more mixing features. For instance, mixing region 109 may comprise a tortuous passage that aids in mixing the components of the curable material. As one embodiment, mixing region 109 may include one or more baffles extending from the wall of mixing region 109 into the lumen of mixing region 109. The baffles may cause turbulence in the flow path of the curable material components to enhance mixing of the components. In other embodiments, mixing region 109 may comprise a static mixer, for example a helical static mixer.

As mentioned previously, in some embodiments the curable material may be delivered through delivery lumen 106 in a solid form. In these embodiments, delivery lumen 106 may include heating element 113, as shown in FIG. 3, proximate distal portion 20 of catheter 10. In these embodiments, delivery lumen 106 may not include any sub-lumens or a mixing region. Rather, when the solid curable material reaches heating element 113, heating element 113 may heat the curable material enough so that the solid curable material transitions to a liquid state. The liquid curable material may then be pushed into second balloon lumen 107 and out ports 35, away from heating element 113. After the liquid curable material has dissipated enough heat, the material will hard again. Additionally, although shown inside delivery lumen 106, the location of heating element 113 in other embodiments may vary. Generally, heating element 113 may be placed anywhere on catheter 10 such that it may heat the solid curable material to a liquid stage so that the curable material may pass through ports 35.

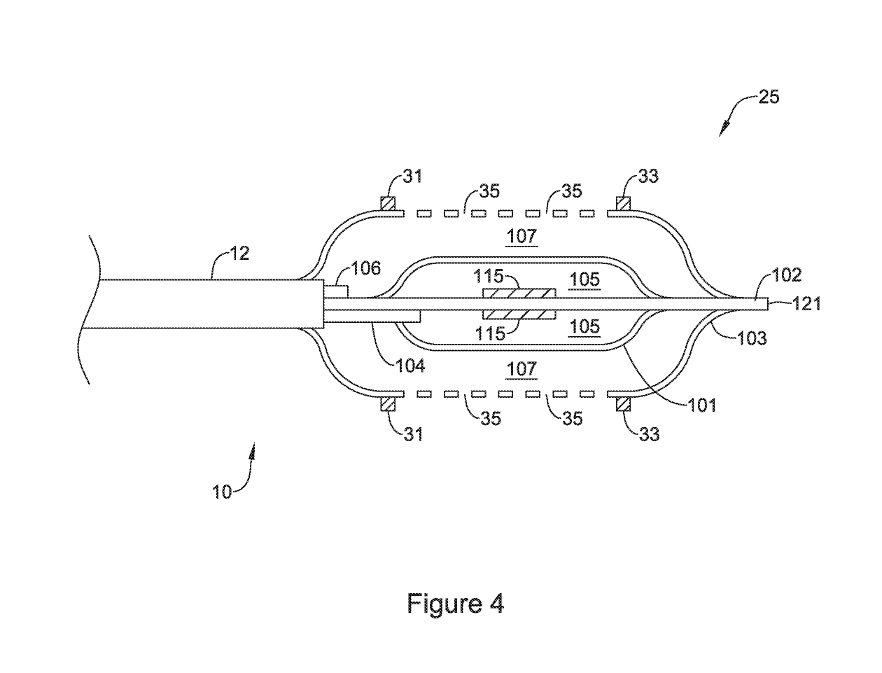

FIG. 4 depicts another embodiment catheter 10, including elongate shaft 12, first balloon member 101, second balloon member 103, first balloon lumen 105, second balloon lumen 107, lumens 102, 104, 106, first raised portion 31, second raised portion 33, and ports 35. The embodiment of FIG. 4 additionally includes energy delivery element 115. In some embodiments, a curing reaction of the curable material may be aided by delivering energy to the curable material. For instance, curable materials that include only one component may benefit from application of light energy, heat energy, and/or RF energy in order to begin or speed up the curing process. Even in some of the embodiments that use two or more curable material components, the curing reaction of the curable material components after mixture may also be aided by applicant of light energy, heat energy, and/or RF energy. Accordingly, energy delivery element 115 may represent an element such as a heating coil to deliver heat energy. The heating coil may comprise a highly resistive material that, when coupled to a source of electricity, converts the electrical energy to heat energy. However, in other embodiments, energy delivery element 115 may represent any suitable heat source. Alternatively, energy delivery element 115 may represent a light source to deliver light energy. For instance, energy delivery element 115 may represent the end of a fiber optic cable that runs at least partially through catheter 10 for delivering light. In some embodiments, the curable material may be curable under UV light. Accordingly, energy delivery element 115 may deliver UV light energy. In other embodiments, energy delivery element 115 may represent any suitable light energy source. In still other embodiments, energy delivery element 115 may represent an RF energy source. For instance, energy delivery element 115 may be an antenna tuned to radiate RF energy when supplied with electrical energy.

In still some additional embodiments, the curable material may cure or harden, or cure or harden more quickly, with the aid of moisture. In some of these embodiments, delivering the curable material through ports 35 into the aqueous environment of the body may cause the curable material to cure and harden. In other embodiments, catheter 10 may include one or more ports dedicated to water delivery. For instance, elongate shaft 12 may include one or more lumens in addition to lumens 102, 104 and 106. The one or more additional lumens may connect up to ports disposed on second balloon member 103, or in other embodiments on first raised portion 21 and/or second raised portion 23. Once the curable material has been delivered to second balloon lumen 107 and through ports 35, a user may deliver water through the one or more additional ports to mix with the delivered curable material.

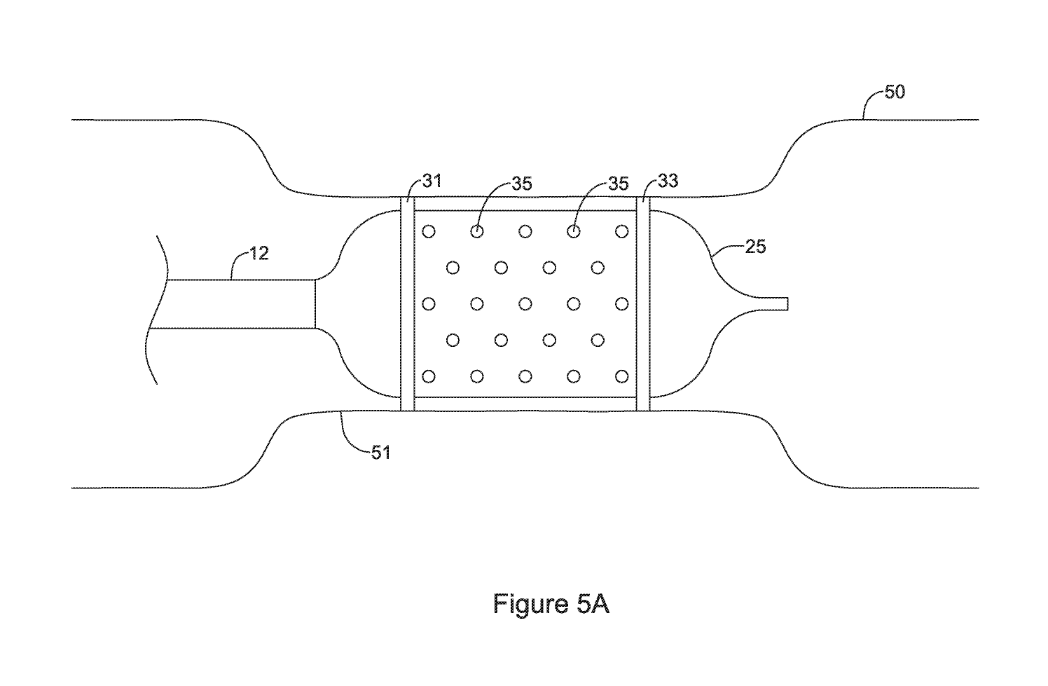

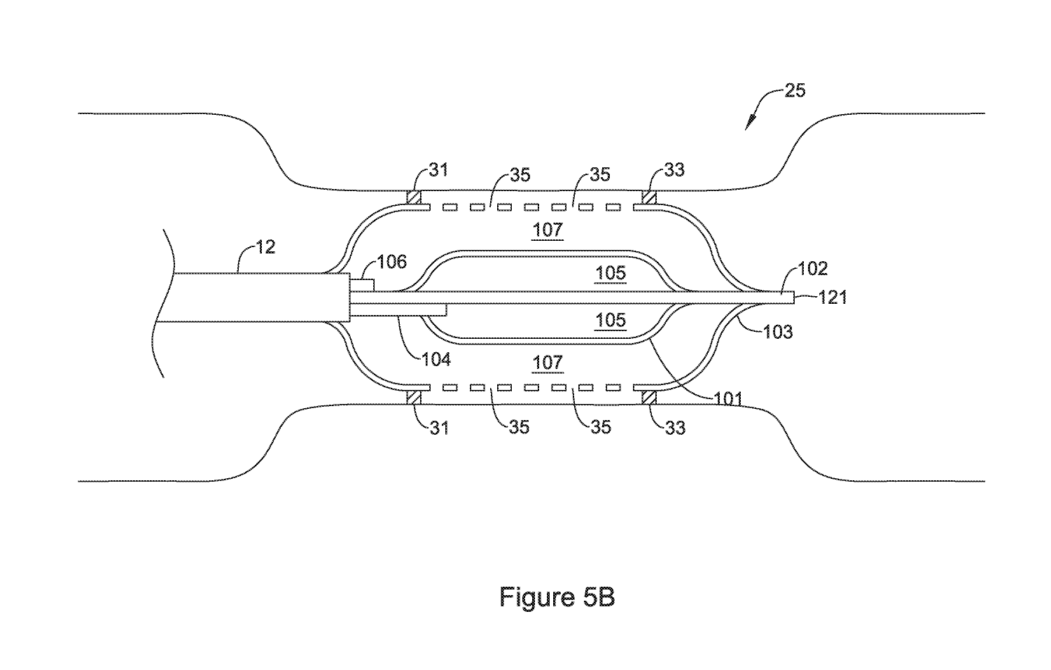

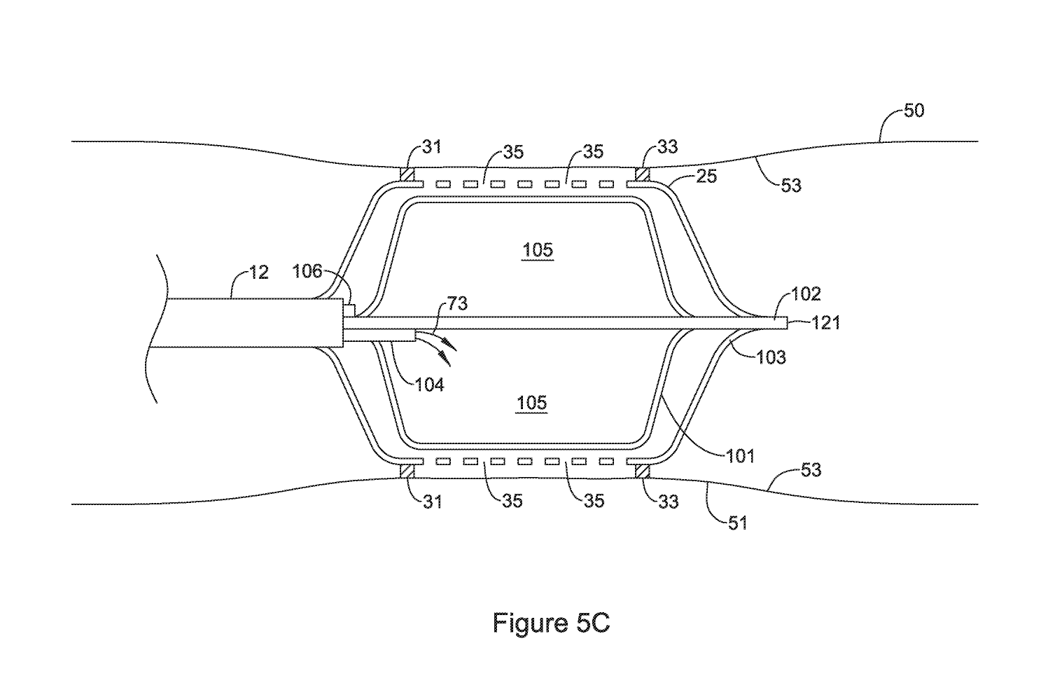

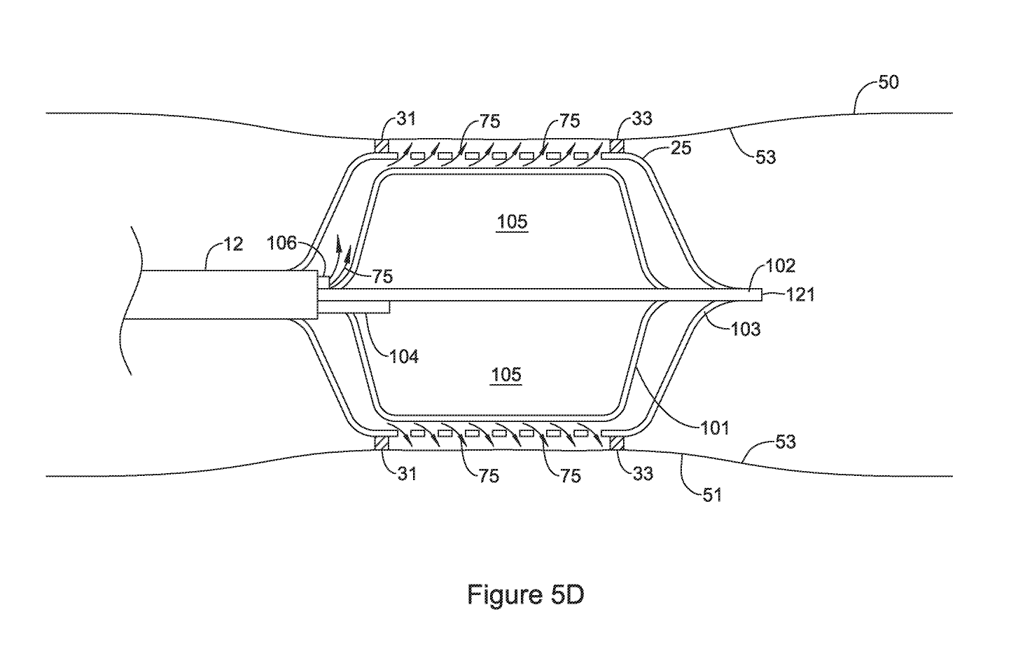

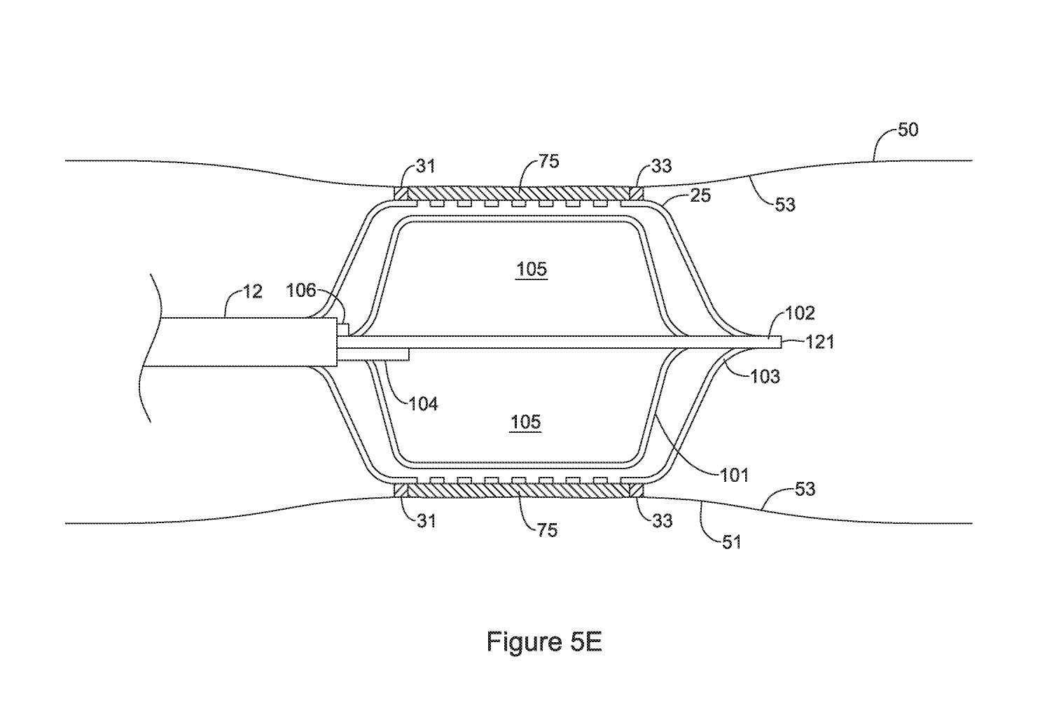

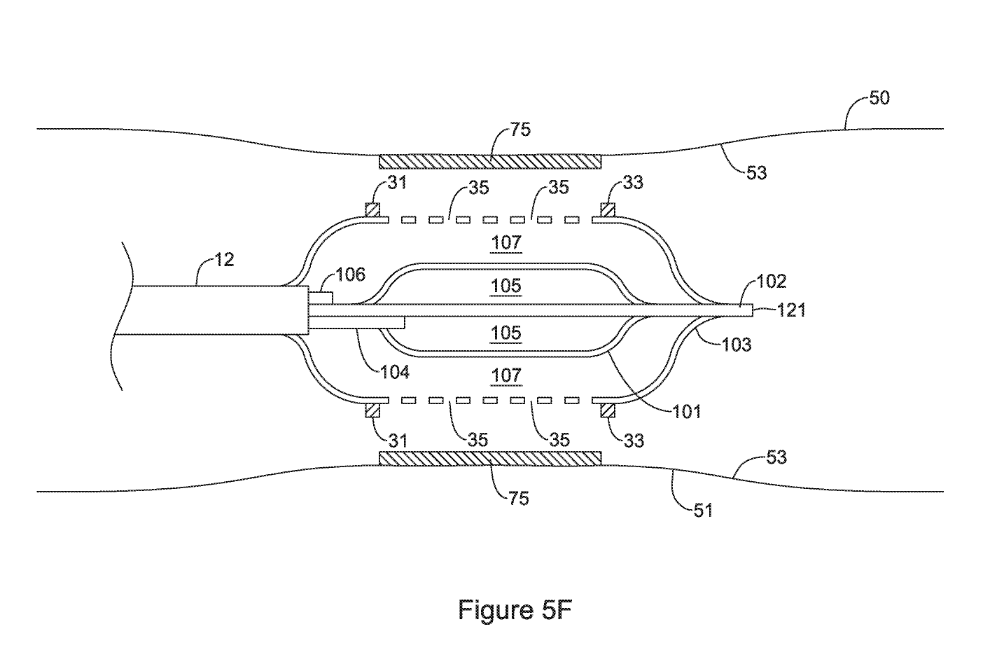

FIGS. 5A-5F depict a method for forming a structured opening using the device described above with respect to FIGS. 1-4. Some patients may suffer from a constriction of a vessel and may find relief by opening the constriction. The device of FIGS. 1-4 may be used to open the constriction and for a structure in-situ that holds the constriction open, as detailed below.

FIG. 5A depicts a portion of elongate shaft 12, along with balloon member 25, including first raised portion 31, second raised portion 33, and ports 35 positioned at constriction 51 of vessel 50. To position balloon member 25 at constriction 51 of biliary vessel 50, a physician may thread balloon member 25 through biliary vessel 50. For instance, a physician may create an opening in biliary vessel 50 away from constriction 51 and insert balloon member 25 into the opening. The physician may then advance balloon member 25 through biliary vessel 50 until balloon member reaches constriction 51 shown in FIG. 5A. In other embodiments, the physician may have first threaded a guidewire through biliary vessel 50 to constriction 51. The physician may then advance balloon member 25 and elongate shaft 12 over the guidewire until balloon member 25 is disposed at constriction 51.

FIG. 5B depicts elongate shaft 12 along with an internal view of balloon member 25, including first balloon member 101 and second balloon member 103 in cross-section, before dilation of constriction 51. FIG. 5B additionally shown lumens 102, 104, 106, first raised portion 31, second raised portion 33, and ports 35, in a similar manner to their depiction in FIG. 2.

Once balloon member 25 is in place at constriction 51, the physician may deliver inflation media 73 through delivery lumen 104 and into first balloon lumen 105 to inflate first balloon member 101, as shown in FIG. 5C. FIG. 5C shows constriction 51 of biliary vessel 50 in a dilated state, with second balloon member 103, including first raised portion 31 and second raised portion 33, pressed against vessel wall 53.

Once first balloon member 101 has been inflated to dilate constriction 51 to a desired size, the physician may then deliver curable material 75 through delivery lumen 106 into second balloon lumen 107, as shown in FIG. 5D. Once a sufficient volume of curable material 75 has been delivered into second balloon lumen 107, curable material 75 may begin to pass through ports 35 to the outside of second balloon member 103. In some embodiments, first raised portion 31 and second raised portion 33, when pressed against a vessel wall, such as vessel wall 53 in FIG. 5C, may be configured to form a seal with vessel wall 53. Accordingly, as curable material 75 passes through ports 35 and outside of second balloon member 103, curable material 75 may be trapped between first raised portion 31, second raised portion 33, and vessel wall 53.

Once a sufficient amount of curable material 75 has been delivered through ports 35, for instance enough curable material 75 to fill the space between first raised portion 31, second raised portion 33, and vessel wall 53, as shown in FIG. 5E, the physician may cease delivering additional curable material 75. The physician may then maintain the position of balloon member 25 while curable material 75 finishes curing and hardens. Where catheter 10 includes an energy delivery element, such as energy delivery element 115 described in FIG. 4, while maintaining the positioning of balloon member 25, the physician may cause the energy delivery element to emit energy to aid the curing reaction, for instance heat energy, light energy, and/or RF energy.

Once hardened, curable material 75 forms a solid, hollow tube, shown in cross-section in FIGS. 5E and 5F. This solid, hollow tube provide resistant to compressive forces acting to constrict biliary vessel 50. Accordingly, hardened curable material 75 maintain patency through biliary vessel 50 and provide relief to the patient.

Once curable material 75 has sufficiently hardened, the physician may siphon inflation media from first balloon lumen 105 to deflate first balloon member 101, as shown in FIG. 5F. In some embodiments, second balloon member 103 may be formed from a material that is adhesion resistant to the curable material so that, when first balloon member 101 is deflated, second balloon member 103 pulls away from the solidified curable material. In other embodiments, the outer surface of second balloon member 103 may be coated with a material that is adhesion resistant to the curable material.

For vessels that contain multiple constrictions, balloon member 25 may be advanced to the next constriction and the method may be repeated. Additionally, or alternatively, for constrictions that extend for a greater length than the length of balloon member 25, after performing the method above, balloon member may be moved just proximal, or just distal of hardened material 75 and the process repeated. In this manner, the device of FIGS. 1-4 may be used to treat constrictions of varying lengths.

Accordingly, the device of FIGS. 1-4 may be used to widen a constriction of a vessel and form a structure in-situ that will maintain an opening through the vessel. This option may have advantages over other dilation methods. For instance, other methods may include using a stent in combination with a dilation balloon. In instances where multiple constrictions need to be treated, multiple deliveries of stents need to be made to the treatment sites which requires insertion and extraction of multiple stent delivery devices. Additionally, stents with open cells may allow for tissue in-growth, which may lead to re-constriction of the site. Alternatively, covered stents may inadvertently block vessel side-branches, which may cause the patient additional or different problems. However, with the device of FIGS. 1-4, when positioned over a vessel side-branch, curable material 75 may simply travel down the vessel side-branch and ultimately be excreted from the patient or absorbed, as the curable material 75 would not be trapped between first raised portion 21, second raised portion 23, and a vessel wall. This would then leave the vessel side-branch patent after hardening of the delivered curable material 75 and the removal of balloon member 25 from the constriction site. Additionally, solidified curable material 75 forms a solid hollow tube, thereby preventing tissue in growth and reconstruction.

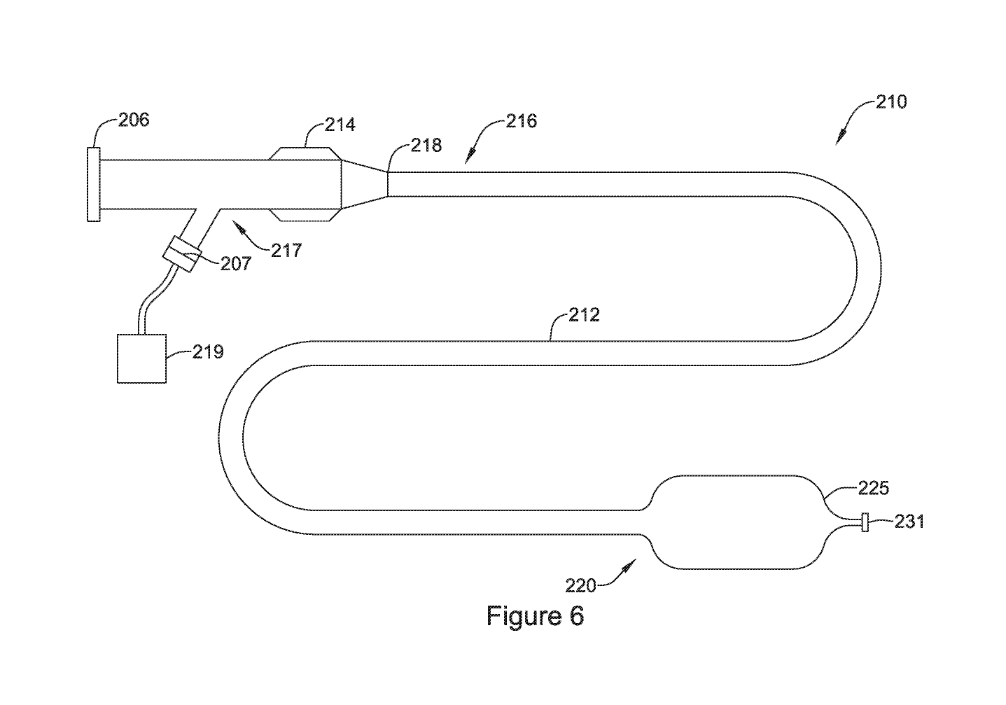

FIG. 6 depicts another embodiment of a device for forming a structured pathway including catheter 210, balloon member 225, with nozzle 231. In some embodiments, catheter 210 may be similar to catheter 10 described with respect to FIG. 1. For instance, in some cases, catheter 210 may be a guide or diagnostic catheter, and may have a length and an outside diameter appropriate for its desired use, for example, to enable biliary tract insertion and navigation. Catheter 210 may be used to navigate to targets sites located in tortuous and narrow vessels such as, for example, to sites within the neurovascular system, certain sites within the coronary vascular system, to sites within the peripheral vascular system such as superficial femoral, popliteal, or renal arteries, or any number of locations within the biliary tract. However, it is contemplated that catheter 210 may be used in other target sites within the anatomy of a patient.

As shown in FIG. 1, catheter 210 can include elongate catheter shaft 212. Elongate shaft 212 may generally extend from proximal portion 216 and proximal end 218 toward distal portion 220. Although elongate shaft 212 may have a circular cross-sectional shape, it should be understood that elongate shaft 212 can have other cross-sectional shapes or combinations of shapes without departing from the scope of the disclosure. For example, the cross-sectional shape of the generally tubular elongate shaft 212 may be oval, rectangular, square, triangular, polygonal, and the like, or any other suitable shape, depending upon the desired characteristics.

In some cases, manifold 214 may be connected to proximal end 218 of elongate shaft 212. The manifold may include hub 217 and/or other structures to facilitate connection to other medical devices (e.g., syringe, stopcocks, Y-adapter, etc.) and to provide access to one or more lumens defined within elongate shaft 212. In some cases, hub 217 may include ports 206 and 207 which provide individual access to one or more lumens extending through at least a portion of catheter 210. Some example lumens that may extend through catheter 210 may include at least one guidewire lumen, one or more inflation lumens, and, in some cases, a lumen for delivering a curable material, whether in a solid form or a liquid form. The lumens that do extend through catheter 210 may terminate at or near distal portion 220 of elongate shaft 212, as will be described with respect to other figures. However, in other cases, hub 217 may have a single port, three ports, or any other number of ports. Manifold 214 may also include a strain relief portion adjacent proximal end 218 of elongate shaft 212.

Distal portion 220 of elongate shaft 212 may include balloon member 225, as shown in FIG. 6. Balloon member 225 may be an inflatable balloon and may have a lumen that is connected to one or more of the lumens extending through elongate shaft 212. For example, balloon member 225 may be a dilation balloon and be connected to an inflation lumen of extending through at least a portion of elongate shaft 212.

In some embodiments, catheter 210 may include nozzle 231 disposed distal of balloon member 225. Nozzle 231 may be disposed at the distal end of one of the lumens that extend at least partially through catheter 210. For instance, at least one of the plurality of lumens of catheter 210 may extend through balloon member 225, and nozzle 231 may be disposed at the distal end of that lumen. Nozzle 231 may regulate the flow of material out of the distal end of catheter 210. For example, as will be described in more detail below, nozzle 231 may have a closed position and an open position and may restrict flow of material out of catheter 210 when in the closed position and may allow flow of material out of catheter 210 when in the open position.

Balloon member 25 may additionally have first raised portion 31 disposed proximate proximal end 32 of balloon member 25. Balloon member 25 may further include second raised portion 33 disposed proximate distal end 34 of balloon member 25. In some embodiments, first raised portion 31 and second raised portion 33 extend all the way around balloon member 25, for example forming ring-like structures.

Catheter 10 may additionally be connected to reservoir 219. Reservoir 219 may be connected to a port of catheter 210, such as port 207, which connects with one or more lumens of catheter 210. Accordingly, reservoir 219 may contain material to be delivered through one or more lumens of catheter 210 to nozzle 231, which regulates the passage of the material out of catheter 210. Reservoir 219 may additionally contain, or be connected to, a pressure source for actively delivering the material stored in reservoir 219 to catheter 210 and nozzle 231. For instance, reservoir 219 may contain, or be connected to, an electric pump that pumps the material into catheter 210. In other embodiments, reservoir 219 may contain, or be connected to, a manual pump that a user may employ to pump the material into catheter 210. In some embodiments, reservoir 219 may represent a syringe filled with the material, where application of force to the syringe plunger pushes the material into and through catheter 210. These are just some examples. Other embodiments may have different sources of pressure.

In accordance with techniques described herein in more detail, catheter 210, including nozzle 231, may be used to form a structure in-situ. For instance, reservoir 219 may contain a curable material that may be delivered to nozzle 231 through a lumen of catheter 210 that is configured to transport the curable material. When positioned at a constriction site, balloon member 225 may be inflated to dilate constriction. Then, curable material may be delivered to nozzle 231 and sprayed on the vessel wall of the constriction site. The curable material may then harden, forming a solid hollow tube resistant to compressive forces. The solid hollow tube may act to maintain patency through the constriction site.

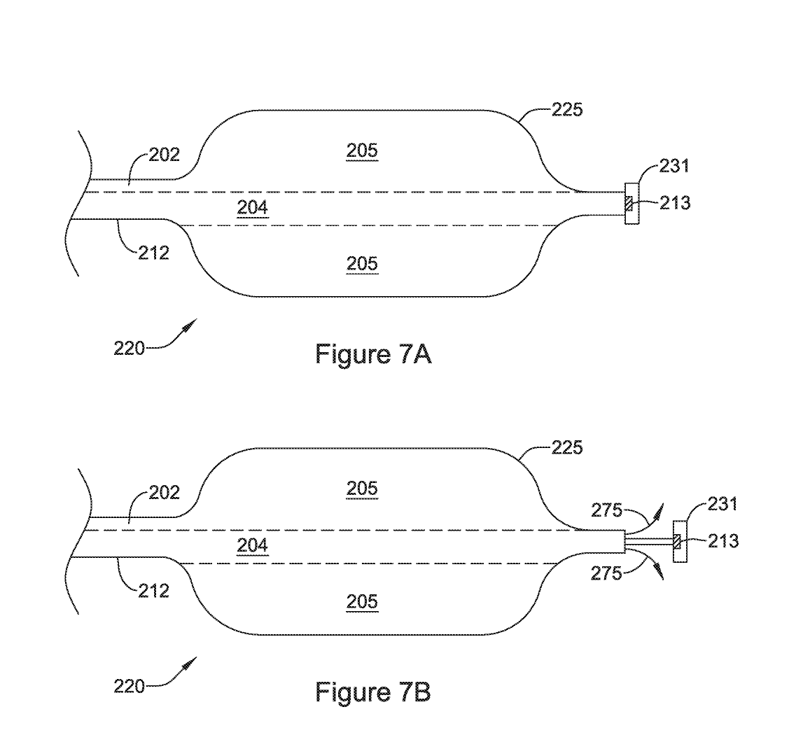

FIG. 7A depicts distal portion 220 of elongate shaft 212, including nozzle 231 and lumens internal to elongate shaft 212. As described above, elongate shaft 212 may include a plurality of lumens extending at least partially through elongate shaft 212. For example, FIG. 7A depicts balloon member 225 including first lumen 202, which is in fluid communication with balloon lumen 205, and second lumen 204, which extends all the way through balloon member 225. Nozzle 231 is situated at the distal end of second lumen 204. When in the closed position, nozzle 231 may prevent flow of material out of second lumen 204. Although not explicitly shown in FIG. 7A, catheter 210 may additionally include a guidewire lumen, for instance in embodiments where catheter 210 may be delivered to a treatment site in an over-the-wire manner. In these embodiments, the guidewire lumen may extend through balloon member 225 in a similar manner as second lumen 204. The guidewire lumen may have a port disposed proximate nozzle 231.

In at least some embodiments, second lumen 204 may be connected to a source of curable material, such as reservoir 219 as depicted in FIG. 6, and configured to deliver the curable material from the curable material source to nozzle 231. Nozzle 231, then, may regulate the flow of curable material out of second lumen 204. As mentioned previously, in different embodiments, the curable may take on many different forms and have many different properties. For instance, the curable material may comprise two or more separate components. Accordingly, in some embodiments, second lumen 204 may be split into two or more sub-lumens and may additionally include a mixing region that is disposed proximal of nozzle 231. In other embodiments, the curable material may be delivered to nozzle in a solid form. In these embodiments, as shown in FIG. 7A, nozzle 231 may additionally include heating element 213. Heating element 213 my melt the solid curable material into a liquid form more suitable for delivery to a vessel wall. Although shown disposed on nozzle 231, in other embodiments, heating element 213 may be disposed at any suitable location within second lumen 204 to melt the delivered curable material.

FIG. 7B depicts elongate shaft 212 and balloon member 225, including nozzle 231 in the open position. When in the open position, curable material 275 may be free to flow through second lumen 204 and out of nozzle 231. In some embodiments, nozzle 231 may be configured to transition from its closed position to its open position when pressure inside second lumen 204 reaches a threshold pressure. Example values for the threshold pressure may range from about 5.0 psi (34.5 kPa) to about 15.0 psi (103 kPa). Accordingly, in these embodiments, curable material 275 may leave nozzle under pressure forming a spray.

It should be understood that nozzle 231 shown in FIGS. 7A and 7B is only a general, exemplary depiction of a nozzle that may be used with catheter 210. For instance, in the example of FIG. 7B, curable material 275 is shown flowing out of second lumen 204 while nozzle 213 has been moved away from second lumen 204 so as not to plug the distal end of second lumen 204. However, in other embodiments, curable material 275 may flow through nozzle 231. For instance, nozzle 231 may have an internal port that may transition from a closed state to an open state. Additionally, nozzle 231 may have an internal configuration that forms a desired spray shape of curable material 275 when curable material 275 exits nozzle 231.

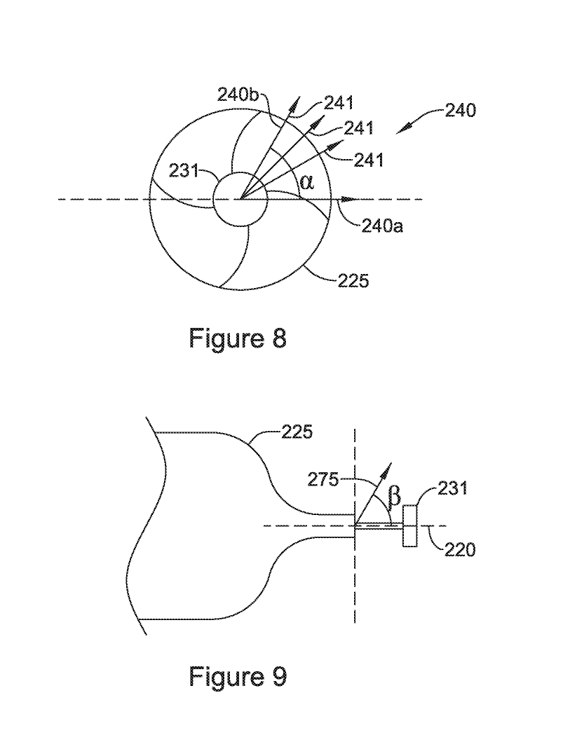

FIG. 8 depicts an end view of balloon member 225 and nozzle 231. In some embodiments, nozzle 231 may be configured to spray curable material 275 away from nozzle 231 in a fan-shaped spray, forming fan-shape 240, as indicated by arrows 241 in FIG. 8. In these embodiments, the fan-shaped spray may form an arc that defines angle α between first edge 240a and second edge 240b of fan-shape 240. In various embodiments, angle α may have a value that ranges from about 30 degrees all the way to about 360 degrees. In more specific embodiments, angle α may have a value that ranges from about 90 degrees to about 180 degrees, or about 180 degrees to about 360 degrees. In still more specific embodiments, angle α may have a value of 45 degrees, 90 degrees, 180 degrees, or 360 degrees.

FIG. 9 depicts a side view of balloon member 225 and nozzle 231. In some embodiments, in addition to, or alternatively to, curable material 275 forming a fan-shaped spray as curable material 275 exits nozzle 231, curable material 275 may exit nozzle 231 at an angle relative to nozzle 231. For example, curable material 275 may form angle θ with to longitudinal axis 220 running through nozzle 231. In various embodiments, angle θ may have a value that ranges from about 10 degrees to about 170 degrees. Accordingly, the spray of curable material 275 may be angled to spray in a distal direction relative to nozzle 231 or in a proximal direction relative to nozzle 231. In more specific embodiments, angle θ may range from about 45 degrees to about 135 degrees. In still more specific embodiments, angle β may have a value of 45 degrees, 90 degrees, or 135 degrees. In at least some embodiments, angle β may be adjustable by a user. For instance, while curable material 275 is being delivered to nozzle 231, a user may adjust angle θ in order to spray curable material 275 over a range of distances from nozzle 231.

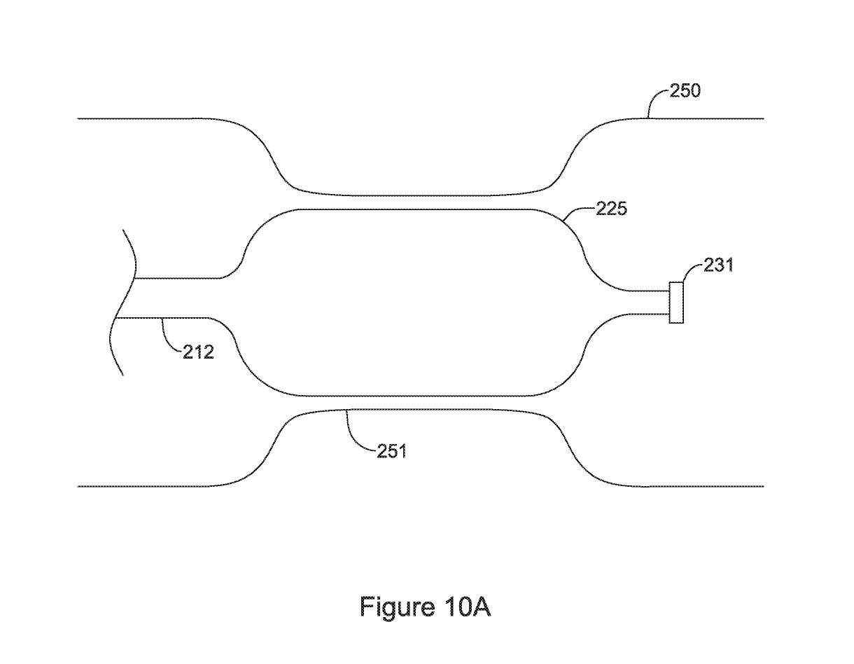

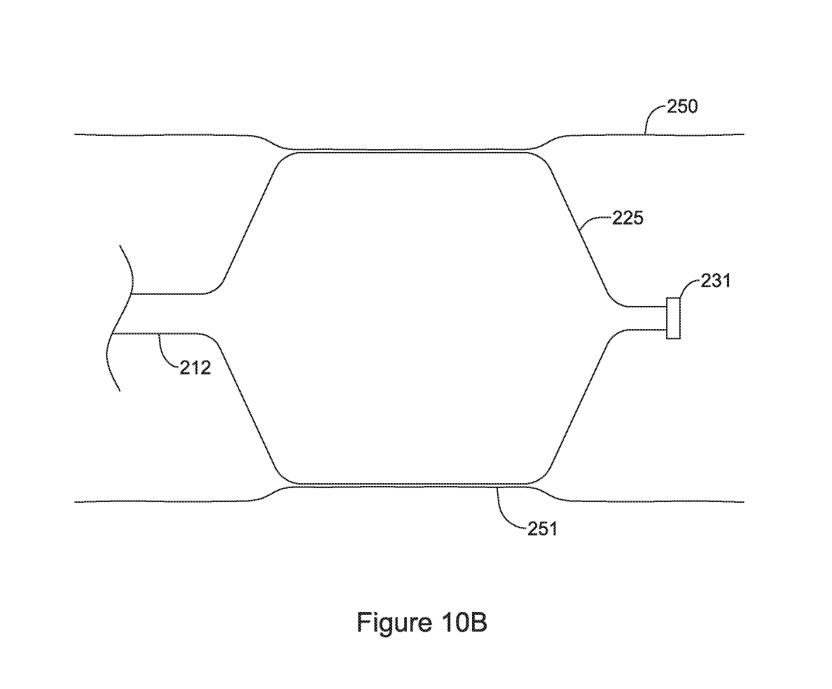

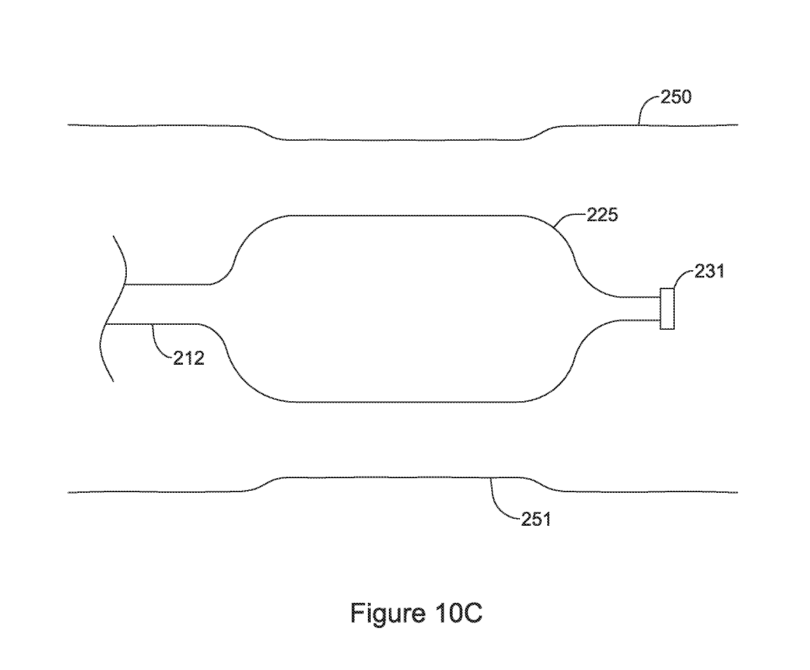

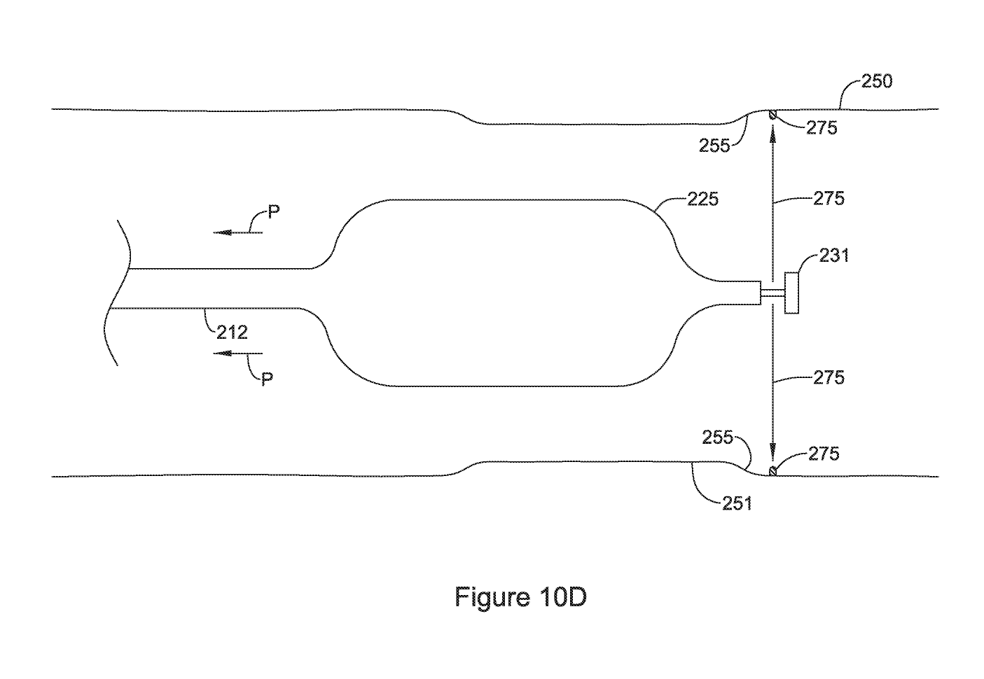

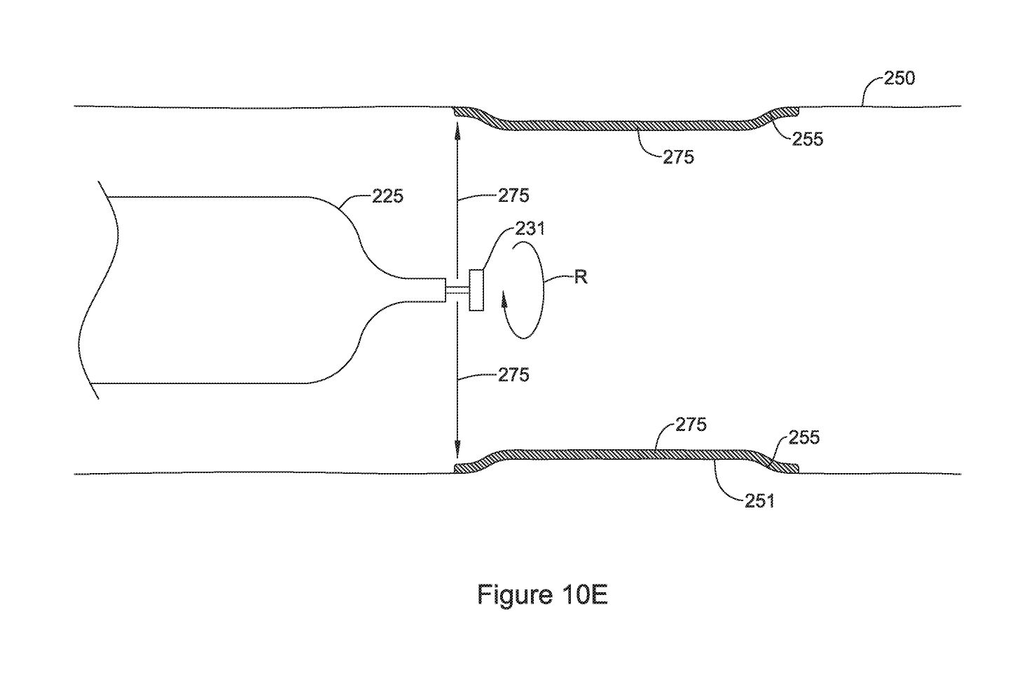

FIGS. 10A-10E depict a method for forming a structured opening using the device described above with respect to FIGS. 6-9. Some patients may suffer from a constriction of a vessel and may find relief by opening the constriction. The device of FIGS. 6-9 may be used to open the constriction and for a structure in-situ that holds the constriction open, as detailed below.

FIG. 10A depicts a portion of elongate shaft 212, along with balloon member 225, including 231 positioned at constriction 251 of vessel 250. To position balloon member 225 at constriction 251 of biliary vessel 250, a physician may thread balloon member 225 through biliary vessel 250. For instance, the physician may create an opening in biliary vessel 250 away from constriction 251 and insert balloon member 225 into the opening. The physician may then advance balloon member 225 through biliary vessel 250 until balloon member reaches constriction 251 shown in FIG. 10A. In other embodiments, the physician may have first threaded a guidewire through biliary vessel 250 to constriction 251. The physician may then advance balloon member 225 and elongate shaft 212 over the guidewire until balloon member 225 is disposed at constriction 251.

Once balloon member 225 is in place at constriction 251, the physician may deliver inflation media (not shown) through an inflation lumen of catheter 210 and balloon member 225 to inflate balloon member 225, as shown in FIG. 10B. FIG. 10B shows constriction 251 of biliary vessel 250 in a dilated state.

Once balloon member 225 has been inflated to dilate constriction 251 to a desired size, the physician may then deflate balloon member 225, as shown in FIG. 10C. Next, the physician may deliver curable material 275 through a lumen of catheter 210 that is connected to nozzle 231, such as second lumen 204 as shown in FIGS. 7A and 7B. Once the pressure in the curable material delivery lumen reaches a threshold pressure, nozzle 231 may transition from its closed state to its open state and may spray curable material 275 onto vessel wall 255 of biliary vessel 250, as depicted in FIG. 10D. In at least some embodiments, curable material 275 may adhere to vessel wall 255, as shown in FIG. 10D forming a covering on vessel wall 255. While delivering curable material 275 through catheter 210 and nozzle 231 to vessel wall 255, the physician may then withdraw balloon member 225 through constriction 251, for example in the direction indicated by arrows P in FIG. 10D.

As balloon member 225 is pulled through constriction 251, curable material 275 may be continuously sprayed onto vessel wall 255, as shown in FIG. 10E. Once at the end of constriction 251, the physician may cease delivering curable material 275 through the curable material delivery lumen and to nozzle 231. In some embodiments, the physician may then extend balloon member 225 back through constriction 251 while delivering more curable material 275 to nozzle 231 and to vessel wall 255. The physician may continue this process until vessel wall 255 has built up a desired thickness of curable material 275. Additionally in some embodiments, the physician may rotate balloon member 223 and nozzle 231, for example as indicated by arrow R in FIG. 10E, to more adequately coat vessel wall 255.

The curable material that has built up on vessel wall 255 may cure and harden into a solid hollow tube, for instance similar to the solid hollow tube described with respect to FIGS. 5A-5F. In some additional embodiments, catheter 210 may further include an energy delivery element. In these embodiments, the physician may then use the energy delivery element to deliver energy to the curable material 275 disposed on vessel wall 255 to cause curable material 275 to cure or to aid curable material 275 in the curing process. In these embodiments, the physician may extend the portion of catheter 210 containing the energy delivery element through constriction 251 one or more times to deliver the energy to curable material 275.

In this manner, the device of FIGS. 6-9 may be used to widen a constriction of a vessel and form a structure in-situ that will maintain an opening through the vessel. This option may have advantages over other dilation methods. For instance, as mentioned previously, other methods may include using a stent in combination with a dilation balloon, and these methods may have a number of drawbacks. In contrast to these other methods, the device of FIGS. 6-9 may be able to form a structure in-situ without blocking vessel side-branches. For example, curable material 275 may simply be sprayed down the vessel side-branch and ultimately be excreted from the patient or absorbed, as opposed to covering the vessel side-branch. This would then leave the vessel side-branch patent after hardening of the delivered curable material 275 and the removal of balloon member 225 from the constriction site. Additionally, solidified curable material 275 may form a solid hollow tube, which would preventing tissue in growth and reconstruction.

In general, the devices described herein, for instance catheters 10 and 210 and balloon members 25 and 225, may be made from any suitable method, and may vary depending on the specific material or materials chosen. For example, if catheters 10 and 210 and balloon members 25 and 225 may be made from a polymer material, catheters 10 and 210 and balloon members 25 and 225 may be made through extrusion.

The materials that can be used for the various components of the devices and components disclosed herein may vary. For simplicity purposes, the following discussion makes reference to catheters 10 and 210, balloon members 25 and 225, and elongate shafts 12 and 212. However, this is not intended to limit the devices and methods described herein, as the discussion may be applied to other similar tubular members and balloon members and/or components of tubular members or balloon members or other devices disclosed herein.

Catheters 10 and 210, balloon members 25 and 225, and/or elongate shafts 12 and 212 may be made from a polymer (some examples of which are disclosed below), a metal-polymer composite, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of catheters 10 and 210, balloon members 25 and 225, and/or elongate shafts 12 and 212 may also be loaded with, made of, or otherwise include a radiopaque material. Radiopaque materials are understood to be materials capable of producing a relatively bright image on a fluoroscopy screen or another imaging technique during a medical procedure. This relatively bright image aids the user of catheters 10 and 210 in determining locations of portions of the devices. Some examples of radiopaque materials can include, but are not limited to, gold, platinum, palladium, tantalum, tungsten alloy, polymer material loaded with a radiopaque filler (e.g., barium sulfate, bismuth subcarbonate, etc.), and the like.

In some embodiments, a coating may be applied to the exterior surface of the catheters 10 and 210, balloon members 25 and 225, and/or elongate shafts 12 and 212. For example, a lubricious, a hydrophilic, a protective, or other type of coating may be applied over portions or all of the catheters 10 and 210, balloon members 25 and 225, and/or elongate shafts 12 and 212. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or catheters 10 and 210, balloon members 25 and 225, and/or elongate shafts 12 and 212, may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

Those skilled in the art will recognize that the present disclosure may be manifested in a variety of forms other than the specific embodiments described and contemplated herein. Specifically, the various features described with respect to the various embodiments and figures should not be construed to be applicable to only those embodiments and/or figures. Rather, each described feature may be combined with any other feature in various contemplated embodiments, either with or without any of the other features described in conjunction with those features. Accordingly, departure in form and detail may be made without departing from the scope of the present disclosure as described in the appended claims.

What is claimed:

1. A medical device for forming a structured pathway in a vessel of a patient, the device comprising:
a catheter shaft extending from a proximal end to a distal end, the catheter shaft including a plurality of catheter shaft lumens extending through at least a portion of the catheter shaft;
a first balloon member disposed proximate the distal end of the catheter shaft, the first balloon member defining a first lumen in fluid communication with a first one of the plurality of catheter shaft lumens; and
a second balloon member disposed proximate the distal end of the catheter shaft and having a proximal end and a distal end, the second balloon member defining a second lumen in fluid communication with a second one of the plurality of catheter shaft lumens,
wherein:
the second balloon member is disposed around the first balloon member,
the second balloon member comprises a plurality of ports disposed on an outer surface of the second balloon member, and
the second balloon member comprises a first raised portion disposed proximate the proximal end of the second balloon member and a second raised portion disposed proximate the distal end of the second balloon member.

2. The medical device of claim 1, wherein the first raised portion extends outward from the outer surface of the second balloon member between about 1.0 mm and about 10.0 mm.

3. The medical device of claim 1, wherein the second raised portion extends outward from the outer surface of the second balloon member between about 1.0 mm and about 10.0 mm.

4. The medical device of claim 1, wherein the first raised portion and the second raised portion extend circumferentially around the second balloon member, each of the first raised portion and the second raised portion forming a ring-like member.

5. The medical device of claim 1, wherein the first raised portion and the second raised portion are configured to create a seal when pressed against an interior wall of the vessel.

6. The medical device of claim 1, wherein the plurality of ports are disposed on the outer surface of the second balloon member between the first raised portion and the second raised portion.

7. The medical device of claim 1, wherein the second one of the plurality of catheter shaft lumens is configured to connect to a source of curable liquid material.

8. The medical device of claim 7, wherein the plurality of ports allow movement of the curable liquid material across the plurality of ports from the lumen of the second balloon member to outside of the second balloon member and restrict movement of the curable liquid material across the plurality of ports from outside of the second balloon member into the lumen of the second balloon member.

9. The medical device of claim 7, wherein an outer surface of the second balloon member is resistant to adhesion by the curable liquid material.

10. The medical device of claim 7, wherein the second of the plurality of lumens is configured to transport curable liquid material from a source of curable liquid material to the lumen of the second balloon member.

11. The medical device of claim 7, further comprising a pressure source for delivering the curable liquid material through the second one of the plurality of catheter shaft lumens and out the plurality of ports.

12. A method of forming a structured pathway in a vessel of a patient, the method comprising:
positioning a medical device at a treatment site within the vessel, the medical device comprising:
a catheter shaft extending from a proximal end to a distal end, the catheter shaft including a plurality of catheter shaft lumens extending through at least a portion of the catheter shaft;
a first balloon member disposed proximate the distal end of the catheter shaft, the first balloon member defining a first lumen in fluid communication with a first one of the plurality of catheter shaft lumens; and
a second balloon member disposed proximate the distal end of the catheter shaft and having a proximal end and a distal end, the second balloon member defining a second lumen in fluid communication with a second one of the plurality of catheter shaft lumens,
wherein:
the second balloon member is disposed around the first balloon member,
the second balloon member comprises a plurality of ports disposed on an outer surface of the second balloon member, and
the second balloon member comprises a first raised portion disposed proximate the proximal end of the second balloon member and a second raised portion disposed proximate the distal end of the second balloon member;
inflating the first balloon member to dilate a portion of the vessel;
delivering a curable liquid material to the lumen of the second balloon member, the curable liquid material exiting the lumen of the second balloon member through the plurality of ports;
maintaining the positioning of the medical device while the curable liquid material solidifies, the solidified curable material forming a structure to maintain an opening through the vessel; and
deflating the first balloon member to separate the first balloon member from the solidified curable material.

13. The method of claim 12, further comprising delivering one or more of light, heat, and RF energy to the curable liquid material to solidify the curable liquid material.

14. The method of claim 12, wherein, when the first balloon member is inflated, the first raised portion and the second raised portion form a seal with an inner wall of the vessel and prevent the curable liquid material exiting the plurality of ports from migrating proximal of the first raised portion and distal of the second raised portion.

15. The method of claim 12, wherein the vessel is in the biliary tract.

16. A medical device for forming a structured pathway in a vessel of a patient, the device comprising:
a catheter shaft extending from a proximal end to a distal end, the catheter shaft including a plurality of catheter shaft lumens extending through at least a portion of the catheter shaft;
a balloon member disposed proximate the distal end of the catheter shaft, the first balloon member defining a first lumen in fluid communication with a first one of the plurality of catheter shaft lumens; and
a port disposed proximate the balloon member, the port being in fluid communication with a second one of the plurality of catheter shaft lumens, the port further configured for excreting a curable liquid material.

17. The medical device of claim 16, wherein the port comprises a nozzle.

18. The medical device of claim 17, wherein the nozzle has a closed configuration and an open configuration, and
wherein the nozzle is configured to spray a curable liquid material when in the open position.

19. The medical device of claim 16, wherein the balloon member is a first balloon member, and
the medical device further comprising a second balloon member having a proximal end and a distal end, the second balloon member comprising a first raised portion disposed proximate the proximal end of the second balloon member and a second raised portion disposed proximate the distal end of the second balloon member

20. The medical device of claim 19, further comprising a plurality of ports disposed between the first raised portion and the second raised portion.
